(19) United States
(12) Patent Application Publication
(10) Pub. No.: US 2019/0177063 A1
(43) Pub. Date: Jun. 13, 2019

(54) ANTI-CONTAMINATION CONTACT LENS PACKAGE AND METHOD FOR MANUFACTURING THE SAME

(71) Applicant: HON HAI PRECISION INDUSTRY CO., LTD., New Taipei (TW)

(72) Inventors: HSIU-WEN CHIEN, New Taipei (TW); JIAN-CHIAO WANG, New Taipei (TW)

(21) Appl. No.: 15/859,761

(22) Filed: Jan. 2, 2018

(30) Foreign Application Priority Data
Dec. 7, 2017 (CN) 201711287545.5

(51) Int. Cl. B65D 65/40; B65D 85/00; A61L 12/08; B01J 35/00; B01J 21/06; B01J 23/06; B01J 27/04; B01J 23/30; B01J 23/745; B01J 23/14; B01J 23/02; B01J 21/08

(52) U.S. Cl. CPC B65D 65/40; B65D 85/54; A61L 12/086; B01J 35/004; B01J 21/063; B01J 23/06; B65D 2585/545; B01J 23/30; B01J 23/745; B01J 23/14; B01J 23/02; B01J 21/08; B01J 27/04

(57) ABSTRACT

An anti-contamination contact lens package includes a substrate and a photocatalyst film layer formed on the substrate. A method for manufacturing the anti-contamination contact lens package is also disclosed.

### FIELD

The subject matter generally relates to an anti-contamination contact lens package and a method for manufacturing the anti-contamination contact lens package.

### BACKGROUND

Common contact lens package is PP cups which is made from polypropylene. Contact lens is manufactured by sending the contact lens after hydrating to a packaging machine to heat seal, and then a moist sterilization process is necessary. However, it is difficult to control the cleanliness of the PP cups. Therefore, when the contact lens is waiting to be sterilized, it may be contaminated by the PP cups and cause contamination of the final product. Improvement in the art is preferred.

### BRIEF DESCRIPTION OF THE DRAWINGS

Implementations of the present disclosure will now be described, by way of example only, with reference to the attached figures.

FIG. 1 is a cross-sectional view of an exemplary embodiment of an anti-contamination contact lens package according to the present disclosure.

FIG. 2 is a flowchart of a method for manufacturing the anti-contamination contact lens package of FIG. 1.

FIG. 3 is a cross-sectional view of another exemplary embodiment of an anti-contamination contact lens package according to the present disclosure.

FIG. 4 is a flowchart of a method for manufacturing the anti-contamination contact lens package of FIG. 3.

### DETAILED DESCRIPTION OF EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The disclosure is illustrated by way of example and not by way of limitation in the figures of the accompanying drawings. It should be noted that references to “an” or “one” embodiment in this disclosure are not necessarily to the same embodiment, and such references mean “at least one.”

The term “comprising” when utilized, means “including, but not necessarily limited to”; it specifically indicates open-ended inclusion or membership in the so-described combination, group, series, and the like.

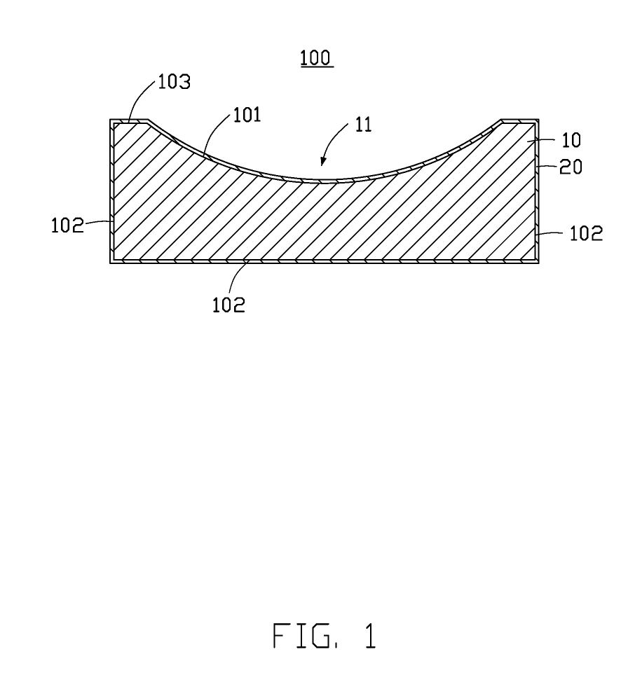

FIG. 1 illustrates an exemplary embodiment of an anti-contamination contact lens package 100. The anti-contamination contact lens package 100 is used to seal contact lenses.

The anti-contamination contact lens package 100 includes a substrate 10 and a photocatalyst film layer 20 formed on the substrate 10.

The substrate 10 includes an inner surface 101, an outer surface 102, and a top surface 103. The top surface 103 connects the inner surface 101 and the outer surface 102. The inner surface 101 is lower than the top surface 103.

In at least one exemplary embodiment, the inner surface 101 is a cured surface.

In at least one exemplary embodiment, the photocatalyst film layer 20 is formed on the inner surface 101, the outer surface 102, and the top surface 103.

The substrate 10 includes a receiving groove 11. The inner surface 101 is an inner wall of the receiving groove 11. The contact lenses are received in the receiving groove 11.

The substrate 10 is a material selected from a group consisting of polypropylene (PP), polyethylene (PE), polycarbonate (PC), polystyrene, and a combination thereof.

In at least one exemplary embodiment, the material of the substrate 10 is PP.

A thickness of the photocatalyst film layer 20 is in a range from 0.003 micrometers to 86 micrometers.

The photocatalyst film layer 20 is made from a photocatalyst material.

The photocatalyst material converts light energy into chemical energy, thereby causing decomposition of organisms (such as bacteria) when irradiated.

When the photocatalyst film layer 20 is irradiated with light having a larger band gap than the photocatalytic film 20, electrons will transit from the valence band to the conduction band, thereby generating electron-hole pairs. The electrons are reducible and the holes are oxidized. The holes will react with OH— on the surface of the photocatalyst film layer 20 to form OH radicals which have strong oxidization. The electrons react with oxygen molecules on the surface of the photocatalyst film layer 20 to form superoxide ion (.O₂). The OH radicals and the superoxide ion decompose microorganisms into carbon dioxide and water, thereby achieving a purifying effect.

Pure photocatalyst material can only absorb ultraviolet light. Photocatalyst material mixed with other active catalytic materials can absorb visible and even far-infrared light.

The photocatalyst material can be selected from a group consisting of titanium dioxide (TiO₂), zinc oxide (ZnO), cadmium sulfide (CdS), tungsten trioxide (WO₃), iron trioxide (Fe₂O₃), lead sulphide (PbS), stannic dioxide (SnO₂), zinc sulfide (ZnS), strontium titanate (SrTiO₃), silicon dioxide (SiO₂), and a combination thereof.

In at least one exemplary embodiment, the photocatalyst material is TiO₂.

In at least one exemplary embodiment, the anti-contamination contact lens package 100 is irradiated by ultraviolet light.

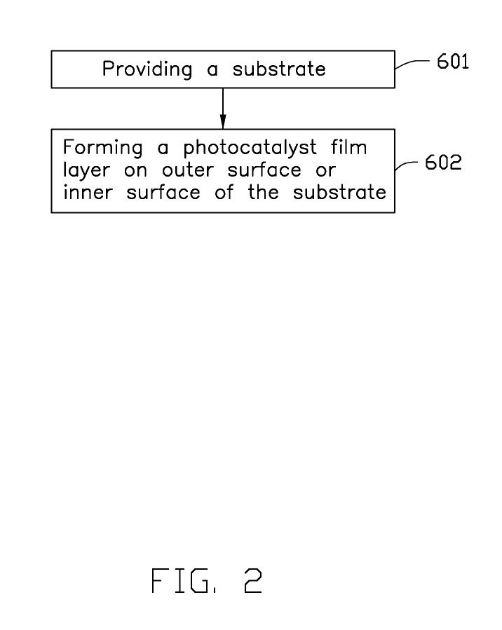

FIG. 2 illustrates a flowchart of a method for manufacturing the anti-contamination contact lens package 100. The method is provided by way of example, as there are a variety of ways to carry out the method. Each block shown in FIG. 2 represents one or more processes, methods, or subroutines, carried out in the exemplary method. Furthermore, the illustrated order of blocks is by example only and the order of the blocks can change. Additional blocks may be added or fewer blocks may be utilized, without departing from this disclosure. The exemplary method can begin at block 601.

At block 601, also illustrated by FIG. 1, a substrate 10 is provided.

The substrate 10 includes an inner surface 101, an outer surface 102, and a top surface 103. The top surface 103 connects the inner surface 101 and the outer surface 102. The inner surface 101 is lower than the top surface 103.

In at least one exemplary embodiment, the inner surface 101 is a cured surface.

The substrate 10 includes a receiving groove 11. The inner surface 101 is an inner wall of the receiving groove 11. The contact lenses are received in the receiving groove 11.

The substrate 10 is a material selected from a group consisting of polypropylene (PP), polyethylene (PE), polycarbonate (PC), polystyrene, and a combination thereof.

In at least one exemplary embodiment, the material of the substrate 10 is PP.

The substrate 10 is made by injection molding.

At block 602, also illustrated by FIG. 1, a photocatalyst film layer 20 is formed on the substrate 10 to form the anti-contamination contact lens package 100.

In at least one exemplary embodiment, the photocatalyst film layer 20 is formed on the inner surface 101, the outer surface 102, and the top surface 103.

A thickness of the photocatalyst film layer 20 is in a range from 0.003 micrometers to 86 micrometers.

The photocatalyst film layer 20 is made from photocatalyst material.

The photocatalyst material converts light energy into chemical energy, thereby causing decomposition of organisms (such as bacteria) when irradiated.

When the photocatalyst film layer 20 is irradiated with light having a larger band gap than the photocatalytic film 20, electrons will transit from the valence band to the conduction band, thereby generating electron-hole pairs. The electrons are reducible and the holes are oxidized. The holes will react with OH— on the surface of the photocatalyst film layer 20 to form OH radicals which have strong oxidization. The electrons react with oxygen molecules on the surface of the photocatalyst film layer 20 to form superoxide ion (.O₂). The OH radicals and the superoxide ion decompose microorganisms into carbon dioxide and water, thereby achieving a purifying effect.

Pure photocatalyst material can only absorb ultraviolet light. Photocatalyst material mixed with other active catalytic materials can absorb visible and even far-infrared light.

The photocatalyst material can be selected from a group consisting of titanium dioxide (TiO₂), zinc oxide (ZnO), cadmium sulfide (CdS), tungsten trioxide (WO₃), iron trioxide (Fe₂O₃), lead sulphide (PbS), stannic dioxide (SnO₂), zinc sulfide (ZnS), strontium titanate (SrTiO₃), silicon dioxide (SiO₂), and a combination thereof.

In at least one exemplary embodiment, the photocatalyst material is TiO₂.

In at least one exemplary embodiment, the anti-contamination contact lens package 200 is irradiated by ultraviolet light.

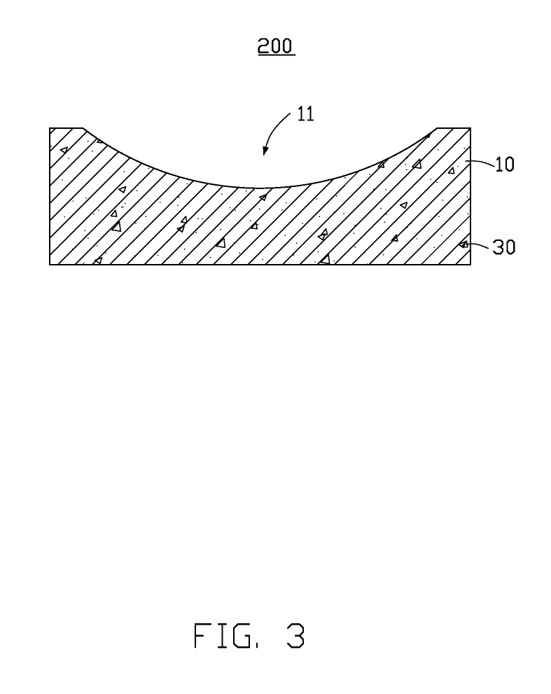

FIG. 3 illustrates another exemplary embodiment of a anti-contamination contact lens package 200. The anti-contamination contact lens package 200 includes a substrate 10 and photocatalyst particles 30 distributed in the substrate 10.

The substrate 10 includes a receiving groove 11. The receiving groove 11 is used to receive contact lenses.

The photocatalyst particles 30 have a mass percentage of about 0.01% to about 13% of the total mass of the anti-contamination contact lens package 200.

The substrate 10 is a material selected from a group consisting of polypropylene (PP), polyethylene (PE), polycarbonate (PC), polystyrene, and a combination thereof.

In at least one exemplary embodiment, the material of the substrate 10 is PP.

The photocatalyst particles 30 are made from photocatalyst material.

The photocatalyst material converts light energy into chemical energy, thereby causing decomposition of organisms (such as bacteria) when irradiated.

When the photocatalyst particles 30 are irradiated with light having a larger band gap than the photocatalyst particles 30, electrons will transit from the valence band to the conduction band, thereby generating electron-hole pairs. The electrons are reducible and the holes are oxidized. The holes will react with OH— on the surface of the photocatalyst particles 30 to form OH radicals which have strong oxidization. The electrons react with oxygen molecules on the surface of the photocatalyst particles 30 to form superoxide ion (.O₂). The OH radicals and the superoxide ion decompose microorganism into carbon dioxide and water, thereby achieving a purifying effect.

Pure photocatalyst material can only absorb ultraviolet light. Photocatalyst material mixed with other active catalytic materials can absorb visible and even far-infrared light.

The photocatalyst material can be selected from a group consisting of titanium dioxide (TiO₂), zinc oxide (ZnO), cadmium sulfide (CdS), tungsten trioxide (WO₃), iron trioxide (Fe₂O₃), lead sulphide (PbS), stannic dioxide (SnO₂), zinc sulfide (ZnS), strontium titanate (SrTiO₃), silicon dioxide (SiO₂), and a combination thereof.

In at least one exemplary embodiment, the photocatalyst material is TiO₂.

In at least one exemplary embodiment, the anti-contamination contact lens package 200 is irradiated by ultraviolet light.

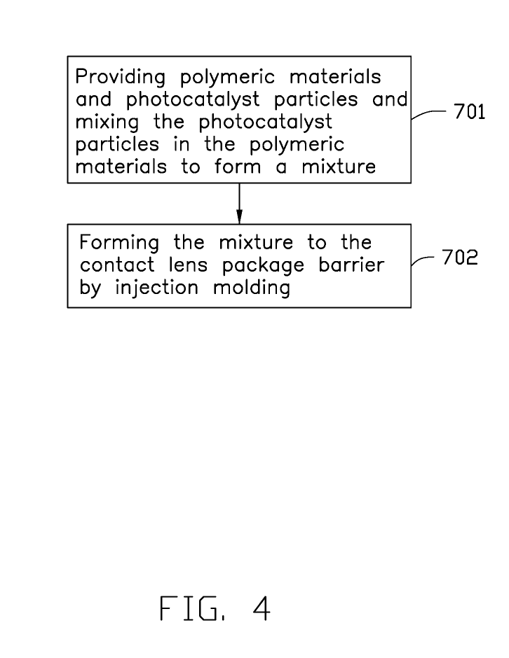

FIG. 4 illustrates a flowchart of a method for manufacturing the anti-contamination contact lens package 200. The method is provided by way of example, as there are a variety of ways to carry out the method. Each block shown in FIG. 4 represents one or more processes, methods, or subroutines, carried out in the exemplary method. Furthermore, the illustrated order of blocks is by example only and the order of the blocks can change. Additional blocks may be added or fewer blocks may be utilized, without departing from this disclosure. The exemplary method can begin at block 701.

At block 701, also illustrated by FIG. 3, polymeric materials and photocatalyst particles 30 are provided and the photocatalyst particles 30 are mixed in the polymeric materials to form a mixture.

The photocatalyst particles 30 have a mass percentage of about 0.01% to about 13% of the total mass of the mixture. The polymeric materials have a mass percentage of about 87% to about 99.99% of the total mass of the mixture.

The polymeric materials can be selected from a group consisting of polypropylene (PP), polyethylene (PE), polycarbonate (PC), polystyrene, and a combination thereof.

In at least one exemplary embodiment, the polymeric materials are PP.

The photocatalyst particles 30 are made from photocatalyst material.

The photocatalyst material converts light energy into chemical energy, thereby causing decomposition of organisms (such as bacteria) when irradiated.

When the photocatalyst particles 30 are irradiated with light having a larger band gap than the photocatalyst particles 30, electrons will transit from the valence band to the conduction band, thereby generating electron-hole pairs. The electrons are reducible and the holes are oxidized. The holes will react with OH— on the surface of the photocatalyst particles 30 to form OH radicals which have strong oxidization. The electrons react with oxygen molecules on the surface of the photocatalyst particles 30 to form superoxide ion (.O₂). The OH radicals and the superoxide ion decompose microorganism into carbon dioxide and water, thereby achieving a purifying effect.

Pure photocatalyst material can only absorb ultraviolet light. Photocatalyst material mixed with other active catalytic materials can absorb visible and even far-infrared light.

The photocatalyst material can be selected from a group consisting of titanium dioxide (TiO₂), zinc oxide (ZnO), cadmium sulfide (CdS), tungsten trioxide (WO₃), iron trioxide (Fe₂O₃), lead sulphide (PbS), stannic dioxide (SnO₂), zinc sulfide (ZnS), strontium titanate (SrTiO₃), silicon dioxide (SiO₂), and a combination thereof.

In at least one exemplary embodiment, the photocatalyst material is TiO₂.

At block 702, also illustrated by FIG. 3, the mixture is injected molding to form the anti-contamination contact lens package 200.

In at least one exemplary embodiment, the anti-contamination contact lens package 200 is irradiated by ultraviolet light.

With the above configuration, the anti-contamination contact lens package 100 has the photocatalyst film layer 20 on the substrate 10 and the anti-contamination contact lens package 200 has the photocatalyst particles 30 distributed in the substrate 10. Because of the photocatalyst film layer 20 and the photocatalyst particles 30 convert light energy into chemical energy, thereby causing decomposition of organisms (such as bacteria) when irradiated, so, the anti-contamination contact lens package 100, 200 can achieve a purifying effect and can avoid being polluted during the making process.

The embodiments shown and described above are only examples. Many details are often found in the art such as the other features of the anti-contamination contact lens package having the same. Therefore, many such details are neither shown nor described. Even though numerous characteristics and advantages of the present disclosure have been positioned forth in the foregoing description, together with details of the structure and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims. It will therefore be appreciated that the embodiments described above can be modified within the scope of the claims.

What is claimed is:

1. An anti-contamination contact lens package, comprising:
a substrate; and
a photocatalyst film layer being formed on the substrate.

2. The anti-contamination contact lens package of claim 1, wherein the substrate includes an inner surface, an outer surface, and a top surface, the top surface connects the inner surface and the outer surface, the inner surface is lower than the top surface; wherein the photocatalyst film layer is formed on the inner surface, the outer surface, and the top surface.

3. The anti-contamination contact lens package of claim 1, wherein the substrate is a material selected from a group consisting of polypropylene, polyethylene, polycarbonate, polystyrene, and a combination thereof.

4. The anti-contamination contact lens package of claim 1, wherein material of the photocatalyst film layer can be selected from a group consisting of titanium dioxide, zinc oxide, cadmium sulfide, tungsten trioxide, iron trioxide, lead sulphide, stannic dioxide, zinc sulfide, strontium titanate, silicon dioxide, and a combination thereof.

5. The anti-contamination contact lens package of claim 1, wherein a thickness of the photocatalyst film layer is in a range from 0.003 micrometers to 86 micrometers.

6. A method for manufacturing an anti-contamination contact lens package, comprising:
providing a substrate; and
forming a photocatalyst film layer on the substrate.

7. The method of claim 6, wherein the substrate is made by injection molding.

8. The method of claim 6, wherein the substrate includes an inner surface, an outer surface, and a top surface, the top surface connects the inner surface and the outer surface, the inner surface is lower than the top surface; wherein the photocatalyst film layer is formed on the inner surface, the outer surface, and the top surface.

9. The method of claim 6, wherein the substrate is a material selected from a group consisting of polypropylene, polyethylene, polycarbonate, polystyrene, and a combination thereof.

10. The method of claim 6, wherein the photocatalyst film layer is formed on the e substrate by electroplating, chemical plating, or pasting.

11. The method of claim 6, wherein a thickness of the photocatalyst film layer is in a range from 0.003 micrometers to 86 micrometers.

12. The method of claim 6, wherein material of the photocatalyst film layer can be selected from a group consisting of titanium dioxide, zinc oxide, cadmium sulfide, tungsten trioxide, iron trioxide, lead sulphide, stannic dioxide, zinc sulfide, strontium titanate, silicon dioxide, and a combination thereof.

13. A anti-contamination contact lens package comprising:
a substrate; and
photocatalyst particles, wherein the photocatalyst particles are distributed in the substrate.

14. The anti-contamination contact lens package of claim 12, wherein the photocatalyst particles have a mass percentage of about 0.01% to about 13% of the total mass of the anti-contamination contact lens package.

15. The anti-contamination contact lens package of claim 12, wherein the substrate is a material selected from a group consisting of polypropylene, polyethylene, polycarbonate, polystyrene, and a combination thereof.

16. The anti-contamination contact lens package of claim 12, wherein material of the photocatalyst particles can be selected from a group consisting of titanium dioxide, zinc oxide, cadmium sulfide, tungsten trioxide, iron trioxide, lead sulphide, stannic dioxide, zinc sulfide, strontium titanate, silicon dioxide, and a combination thereof.

17. A method for manufacturing an anti-contamination contact lens package, comprising:
providing polymeric materials and photocatalyst particles and mixing the photocatalyst particles in the polymeric materials to form a mixture;
forming the mixture to the anti-contamination contact lens package by injection molding.

18. The method of claim 17, wherein the photocatalyst particles have a mass percentage of about 0.01% to about 13% of the total mass of the mixture, the polymeric materials have a mass percentage of about 87% to about 99.99% of the total mass of the mixture.

19. The method of claim 17, wherein the polymeric materials can be selected from a group consisting of polypropylene, polyethylene, polycarbonate, polystyrene, and a combination thereof.

20. The method of claim 17, wherein material of the photocatalyst particles can be selected from a group consisting of titanium dioxide, zinc oxide, cadmium sulfide, tungsten trioxide, iron trioxide, lead sulphide, stannic dioxide, zinc sulfide, strontium titanate, silicon dioxide, and a combination thereof.
